(19) United States
(12) Patent Application Publication
(10) Pub. No.: US 2023/0400429 A1
(43) Pub. Date: Dec. 14, 2023

(54) BIOASSAY DEVICE WITH EVENLY DISPERSED CARRIERS

(71) Applicant: Guangzhou Tyrafos Semiconductor Technologies Co., Ltd., Guangzhou (CN)

(72) Inventors: Hsu-Wen Fu, Kaohsiung City (TW); Jun-Wen Chung, Tainan City (TW); Ping-Hung Yin, Taipei City (TW)

(21) Appl. No.: 17/966,913

(22) Filed: Oct. 17, 2022

Related U.S. Application Data
(60) Provisional application No. 63/350,880, filed on Jun. 10, 2022.

(51) Int. Cl. G01N 27/327; G01N 21/64

(52) U.S. Cl. CPC G01N 27/3275; G01N 21/64; B01L 2400/043; B01L 2300/0816; B01L 2400/0427; B01L 2300/0654

(57) ABSTRACT

A bioassay device with evenly dispersed carriers includes an image sensor unit, a plurality of microstructures and a first EWOD device. The image sensor unit includes a substrate and a plurality of unit pixels, the substrate has a light-receiving surface, the plurality of unit pixels are disposed in the substrate and close to the light-receiving surface, and each unit pixel has a photoelectric conversion unit. The plurality of microstructures is disposed on the light-receiving surface and forms a plurality of grooves, and the plurality of grooves is respectively located above the plurality of unit pixels. The first EWOD device includes a plurality of first EWOD electrodes, the plurality of first EWOD electrodes is disposed on the light-receiving surface outside of the grooves, or the plurality of first EWOD electrodes is disposed above the substrate.

### CROSS-REFERENCE TO RELATED APPLICATIONS

This application claims the priority of U.S. provisional patent application No. 63/350,880, filed on Jun. 10, 2022, which are incorporated herewith by reference.

### BACKGROUND OF THE INVENTION

### 1. Field of the Invention

The present invention relates generally to a bioassay device, and more particularly, to a bioassay device in which the carrier is evenly dispersed.

### 2. The Prior Arts

Enzyme-Linked Immunosorbent Assay (ELISA) or Enzyme-linked immunoassay (EIA) is a so-called specific antigen-antibody reaction test. The specific binding characteristics of the antigen and antibody is used to perform detection on the sample to be tested, and in combination with the enzyme to perform a chemical luminescence reaction (i.e., chemiluminescence), the presence of a specific antigen or antibody can be displayed. Furthermore, the intensity of the chemical luminescence can be quantitatively analyzed, so as to achieve the objective of detection and screening.

A biochip is a microscopic light-sensing device that can produce a specific biochemical reaction with the biomolecules to be tested by placing a biomaterial on a light sensing chip, and can sensitively detect the intensity of the chemical light emitted by the biochemical reaction and convert the light signal into electrical signals. The biochip has the capability of fast, accurate, and low-cost biological analysis and testing. In molecular biology, the biochip can simultaneously sense the chemical light emitted by hundreds or tens of thousands of biochemical reactions.

A microfluidic chip is a biochip in which micron-scale microstructures and/or microfluidic channels are formed on a light-sensing chip, and the direction and volume of a fluid sample are precisely controlled by laminar flow. Microfluidic chips have the following advantages: first, small capacity, saving reagent consumption; second, small size, easy to carry; third, low energy consumption, reducing power supply; fourth, easy to quantify, able to get a lot of data in a short time.

The fluid sample in the microfluidic chip may contain magnetic beads, and the droplets containing the magnetic beads are mixed with the sample to be tested (e.g., sputum, saliva, tissue, whole blood, serum, etc.) in the flow channel. The magnet below the microfluidic chip can make the magnetic beads sink to the surface of the chip by the magnetic force. According to the high specificity of antibody-antigen affinity in the principle of immunology, the antibody on the magnetic bead can bind to the biomolecule in the sample to be tested, by detecting the light emitted by the fluorescent label or chemiluminescence on the biomolecule, the presence and/or concentration of the biomolecule are determined

However, the flow channel can only provide a unidirectional flow of droplets, and the magnetic beads can only be randomly dispersed on the chip surface. Therefore, there are more magnetic beads in some areas, and fewer magnetic beads in some areas, which cannot be evenly dispersed on the chip surface, resulting in a decrease in the accuracy of determining the presence and/or concentration of biomolecules.

Furthermore, the structure of the microfluidic chip is relatively closed, so it has the following two disadvantages: first, the residuals in the flow channel cannot be cleaned, resulting in the incapability of reusability of the microfluidic chip; second, the size of the droplets is limited by the size of the inlet, it is difficult to control the size of the droplet, and it is impossible to control the size of the droplet to limit the number of magnetic beads. It may happen that the number of magnetic beads in each area exceeds expectations, which reduces the judgment of the presence and/or concentration accuracy of biomolecules.

In addition, factors such as the height of the flow channel or the relatively high hydrophilicity of the surface of the flow channel will reduce the smoothness of the movement of the droplets.

Finally, the microfluidic chip is not easy to manufacture, and the manufacturing cost is high.

### SUMMARY OF THE INVENTION

A primary objective of the present invention is to provide a bioassay with evenly dispersed carriers. By controlling the movement of the droplets, the carrier can be evenly dispersed in the groove.

In order to achieve the aforementioned objective, the present invention provides a bioassay with evenly dispersed carriers, including an image sensing element, a plurality of microstructures, and a first electrowetting-on-dielectric device. The image sensing element includes a substrate and a plurality of unit pixels, the substrate has a light-receiving surface, the unit pixels are disposed inside the substrate and close to the light-receiving surface, and each unit pixel has a photoelectric conversion element. The microstructures are disposed on the light-receiving surface and form a plurality of grooves, and the grooves are respectively located above the unit pixels. The first electrowetting-on-dielectric device includes a plurality of first electrowetting-on-dielectric electrodes, the first electrowetting-on-dielectric electrodes are disposed on the light-receiving surface and outside the grooves, or the first electrowetting-on-dielectric electrodes are disposed above the substrate.

The effect of the present invention is that when the first electrowetting-on-dielectric (EWOD) electrodes are energized, the first EWOD electrodes generate an electrostatic force to control a droplet containing a plurality of carriers to move back-and-forth on the light-receiving surface so that the carriers are dispersed evenly in the plurality of grooves, and each carrier carries at least one biomolecule.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is a schematic structural view of a first embodiment of the present invention.

FIG. 2 is a top view of the first embodiment of the present invention.

FIG. 3 is a schematic view of a droplet containing a plurality of carriers, wherein the carriers carry biomolecules.

FIG. 4 is a schematic view of controlling droplet movement according to the first embodiment of the present invention.

FIG. 5 is a schematic view of the carrier evenly dispersed in the grooves of the first embodiment of the present invention.

FIG. 6A is a flowchart of a first method for detecting biomolecules using the first embodiment of the present invention.

FIG. 6B is a flowchart of a second method for detecting biomolecules using the first embodiment of the present invention.

FIG. 7 is a schematic structural view of a second embodiment of the present invention.

FIG. 8 is a schematic structural view of a third embodiment of the present invention.

FIG. 9 is a schematic structural view of a fourth embodiment of the present invention.

FIG. 10 is a schematic view of controlling the movement of droplets according to the fourth embodiment of the present invention.

FIG. 11 is a schematic view of the carrier evenly dispersed in the grooves of the fourth embodiment of the present invention.

FIG. 12A is a schematic structural view of a fifth embodiment of the present invention.

FIG. 12B is a top view of the structure of the fifth embodiment of the present invention.

FIG. 13A is a flow chart for controlling the movement of droplets according to the fifth embodiment of the present invention.

FIG. 13B is a schematic view of controlling droplet movement according to the fifth embodiment of the present invention.

FIG. 14 is a schematic view of the carrier evenly dispersed in the grooves of the fifth embodiment of the present invention.

FIG. 15 is a schematic structural view of a sixth embodiment of the present invention.

FIG. 16 is a schematic view of controlling droplet movement according to the sixth embodiment of the present invention.

FIG. 17 is a schematic structural view of a seventh embodiment of the present invention.

FIG. 18 is a schematic view of controlling the movement of droplets

according to the sixth embodiment of the present invention.

### DETAILED DESCRIPTION OF THE PREFERRED EMBODIMENT

The accompanying drawings are included to provide a further understanding of the invention, and are incorporated in and constitute a part of this specification. The drawings illustrate embodiments of the invention and, together with the description, serve to explain the principles of the invention.

According to the present invention, the electrowetting-on-dielectric (EWOD) is to change the original equilibrium state by applying a voltage difference between the electrolyte droplet and the wall surface. Under this principle, the wall is divided into many blocks, and the voltage difference applied on block is controlled to make the droplet continuously change the equilibrium state to achieve the goal of moving the droplet.

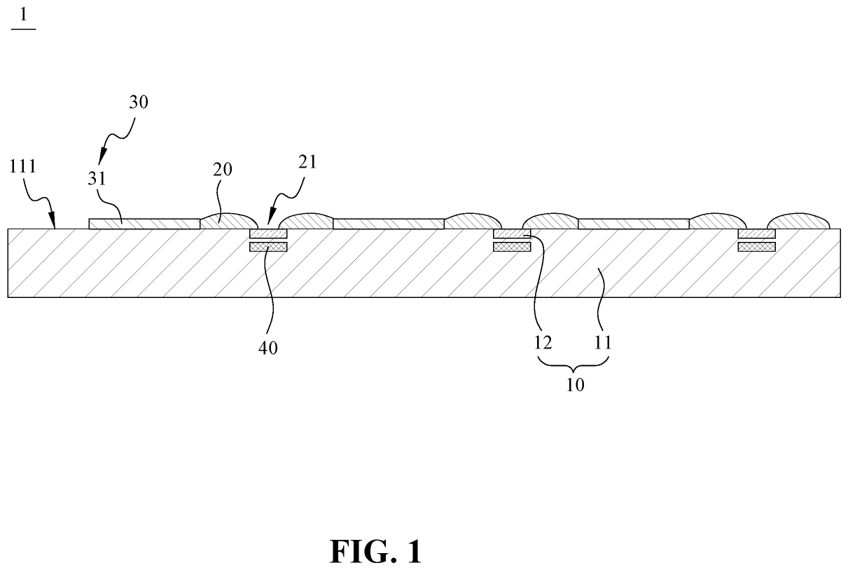

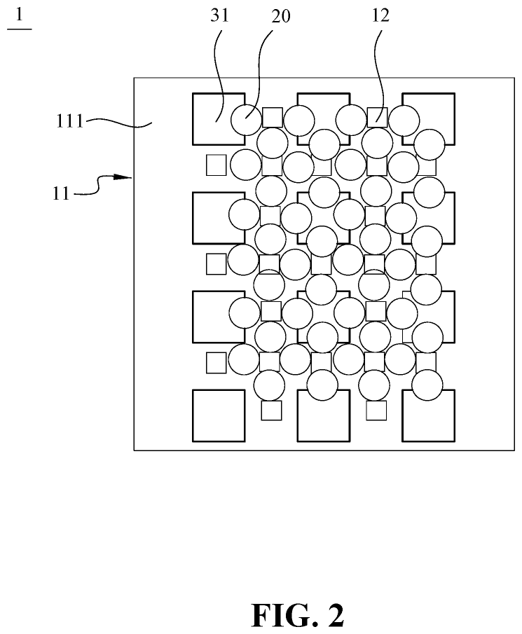

FIG. 1 is a schematic structural view of the first embodiment of the present invention, and FIG. 2 is a top view of the first embodiment of the present invention. As shown in FIG. 1 and FIG. 2, the present invention provides a bioassay 1, which includes an image sensing element 10, a plurality of microstructures 20, and a first electrowetting-on-dielectric (EWOD) device 30. The image sensing element 10 includes a substrate 11 and a plurality of unit pixels 12. The substrate 11 has a light-receiving surface 111, the unit pixels 12 are disposed inside the substrate 11 and close to the light-receiving surface 111, and each unit pixel 12 has a photoelectric conversion element (not shown). The microstructures 20 are disposed on the light-receiving surface 111 and form a plurality of grooves 21, and the grooves 21 are respectively located above the unit pixels 12. The first EWOD device 30 includes a plurality of first EWOD electrodes 31, and the first EWOD electrodes 31 are disposed on the light-receiving surface 111 and outside the grooves 21.

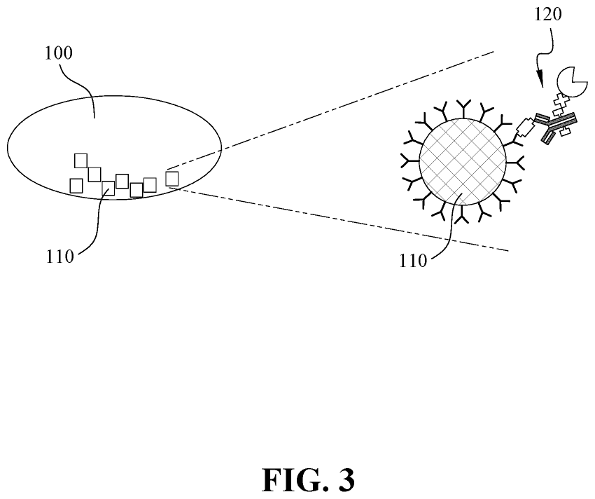

FIG. 3 is a schematic view of a first embodiment of a droplet 100 containing a plurality of carriers 110, wherein the carriers 110 carry biomolecules 120. As shown in FIG. 3, the droplet 100 directly contains a plurality of carriers 110, and each carrier 110 carries at least one biomolecule 120. The biomolecules 120 can be, for example, proteins, peptides, antibodies, nucleic acids, and the like.

Specifically, as shown in FIG. 3, the carrier 110 can be a microparticle, and through the EDC/NHS reaction, the biomolecules 120 such as antibodies are linked with the microparticles by forming amide bonds. The sample to be tested is mixed with antibodies and microparticles so as to capture the antigen to be tested from the sample to be tested. Then, the secondary antibody of the modified biotin is combined with the antigen; the biotin is combined with streptavidin to bring in a plurality of horseradish peroxidase (HRP) molecules, so as to form a microparticle-antibody-antigen-antibody-biotin-streptavidin-polyHRP complex.

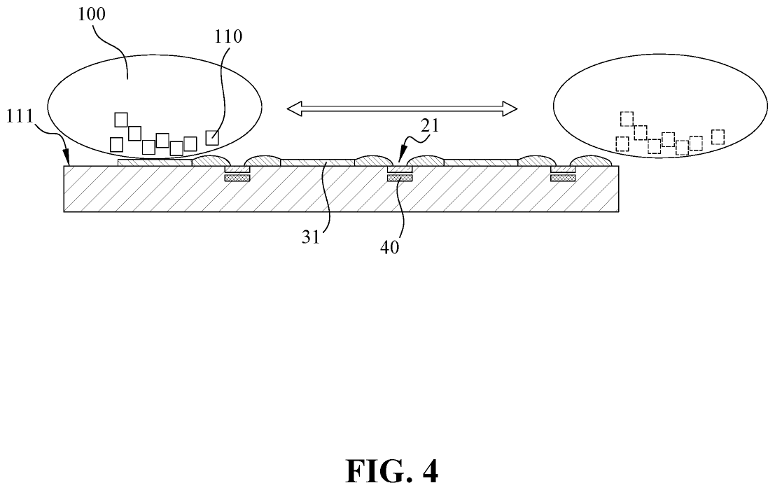

FIG. 4 is a schematic view of controlling the movement of the droplet 100 according to the first embodiment of the present invention. As shown in FIG. 4, when the first EWOD electrodes 31 are energized, the first EWOD electrodes 31 generate an electrostatic force and control the droplet 100 to move back and forth on the light-receiving surface 111 by the electrostatic force.

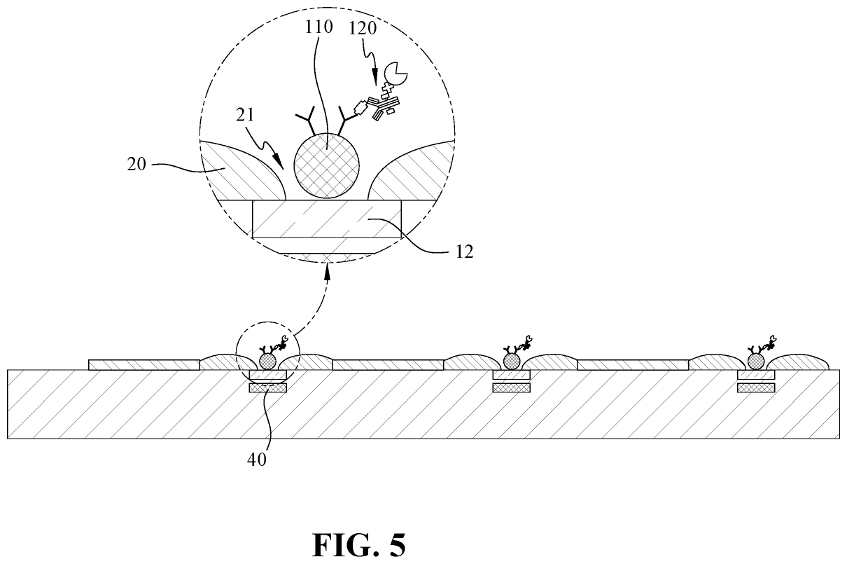

FIG. 5 is a schematic view of the carrier 110 being evenly dispersed in the grooves 21 of the first embodiment of the present invention. As shown in FIG. 4 and FIG. 5, during the process of the droplet 100 moving back and forth on the light-receiving surface 111, the droplet 100 will repeatedly pass through the grooves 21 for many times, so that the carriers 110 are evenly dispersed in the grooves 21, and each carrier 110 carries at least one biomolecule 120.

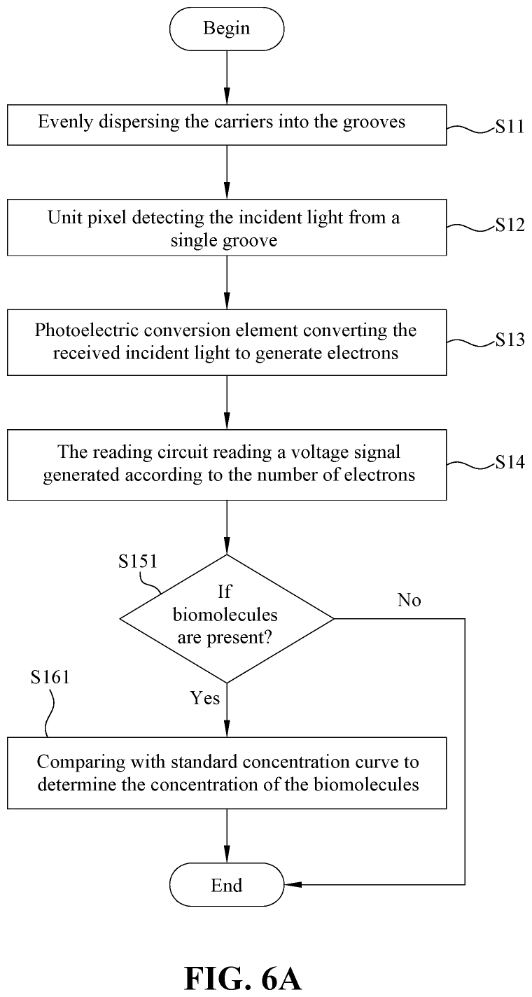

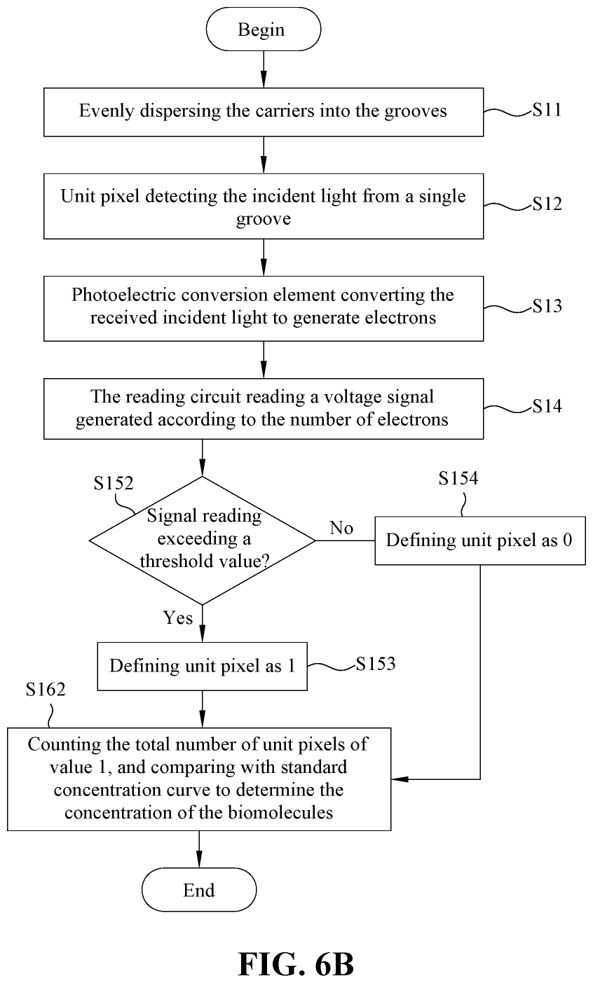

FIGS. 6A and 6B are flowcharts of a first method and a second method for detecting biomolecules 120 using the first embodiment of the present invention, respectively. As shown in FIG. 6A and FIG. 6B , and also refer to FIG. 4 and FIG. 5, the method for detecting biomolecules 120 using the bioassay 1 includes the following steps: in step S11, adding markers, fluorescent labels, reporter molecule labels, or chemiluminescence labels to the biomolecules 120 in the sample to be tested by biological or chemical analysis such as ELISA, and using the electrostatic force of the first EWOD electrodes 31 to control the back and forth movement of the droplets 100, so that the carriers 110 being evenly dispersed in the plurality of grooves 21; in step S12, causing the unit pixels 12 to detect an incident light in a single groove 21; in step S13, the incident light being a light emitted by the markers, fluorescent labels, reporter molecule labels, or chemiluminescence labels of the biomolecules 120, and then the incident light received by each of the unit pixels 12 being transmitted through the photoelectric conversion element to generate electrons; and in step S14, using a plurality of readout circuits (not shown) coupled to the unit pixels 12 to generate a voltage signal according to the number of electrons, and then analyze the presence and/or concentration of the biomolecules 120 according to the voltage signal.

When analyzing the presence and/or concentration of biomolecules 120 using the bioassay device 1, in addition to being quantified by analog colorimetry, that is, with the incident light a single groove 21 received by each of the unit pixels 12 as a single signal reading to determine whether the biomolecule 120 exists (step S151), or compare it with the standard concentration curve to obtain the concentration of the biomolecule 120 (step S161); digital methods can also be used to perform quantification, that is, according to the set threshold value (step S152), defining the unit pixel 12 with measured signal reading value exceeding the threshold value as 1 (step S153), and defining the unit pixel 12 with measured signal reading value not exceeding the threshold value as 0(step S154); finally, calculating the total number of unit pixels 12 as 1 and comparing with the standard concentration curve (step S162), so as to obtain the more accurate concentration of the biomolecule 120.

It is worth mentioning that the first EWOD electrodes 31 are disposed outside of the grooves 21 and will neither prevent the carriers 110 from entering the grooves 21 nor affect the unit pixels 12 detecting the incident light in a single groove 21, respectively.

In addition, the structure of the bioassay 1 is relatively open, and has the following two advantages: first, the attachments on the outer surfaces of the microstructures 20, the top surfaces of the unit pixels 12, the outer surfaces of the first EWOD electrodes 31, and the surface and the exposed light-receiving surface 111 can be cleaned, so that the bioassay 1 can be reused; second, the size of the droplet 100 is easy to control, and the size of the droplet 100 is controlled to limit the number of carriers 110 to be lower than the number of grooves 21, so that each groove 21 has only one carrier 110 at most, that is, each unit pixel 12 can only detect the incident light of the biomolecules 120 of one carrier 110, which improves the accuracy of determining the presence and/or concentration of the biomolecule 120.

As shown in FIG. 1, in the first embodiment, the bioassay 1 further includes a plurality of magnets 40. The magnets 40 are arranged inside the substrate 11 and are respectively arranged below the grooves 21. The range of the magnetic force of each magnet 40 covers each groove 21. As shown in FIG. 3, the microparticles are preferably magnetic beads of 1 to 3 μm, and the magnetic beads may use magnetic elements such as iron (Fe), nickel (Ni), cobalt (Co), etc., ferromagnetic alloys, such as neodymium iron boron (Nb—Fe—B), or magnetic materials of iron oxides such as triiron tetroxide (Fe3O4), iron oxide (Fe2O3), and ferrous oxide (FeO). As shown in FIG. 4 and FIG. 5, during the process of the droplet 100 moving back and forth on the light-receiving surface 111, the droplet 100 will repeatedly pass through the grooves 21, and the magnetic force of the magnets 40 will attract the magnetic beads down to sink into the grooves 21, so that the magnetic beads are evenly dispersed in the grooves 21.

In some embodiments, the microparticles can also use non-magnetic materials, such as gold (Au), sepharose, polystyrene, silicon dioxide (SiO2), so the bioassay device of these embodiments does not include magnets 40.

As shown in FIGS. 1 and 2, in the first embodiment, each microstructure is a microlens. In some embodiments, each microstructure 20 exhibits various possible geometric shapes, such as honeycomb-like or inverted pyramid-shape.

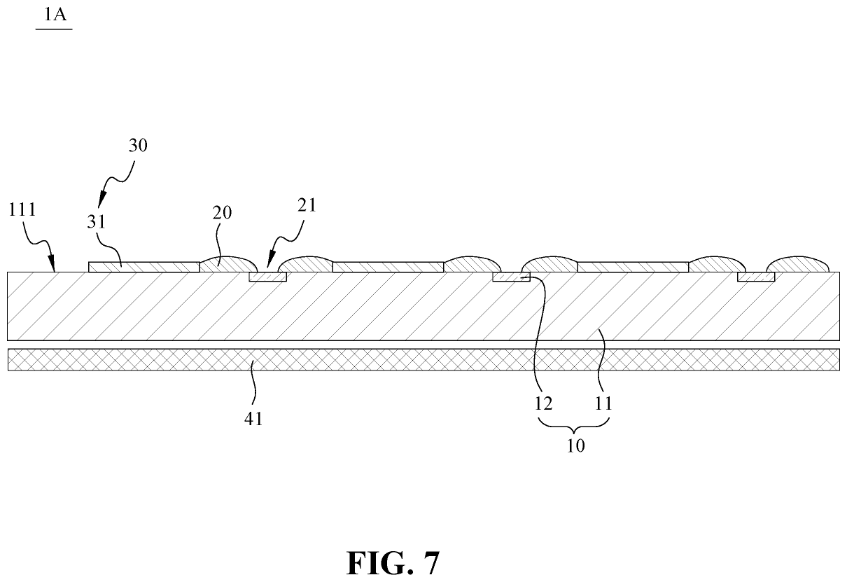

FIG. 7 is a schematic structural view of a second embodiment of the present invention. As shown in FIG. 7, the difference between the bioassay 1A of the second embodiment and the bioassay 1 of the first embodiment is that the bioassay 1A includes a magnet 41. The magnet 41 is disposed under the substrate 11 and has a magnetic force range that covers grooves 21. Compared with the first embodiment, because the size of the substrate 11 is smaller, the bioassay 1A of the second embodiment disposes a magnet 41 with a larger size below the substrate 11, which is less difficult to manufacture and reduces the manufacturing cost, while achieving the same effect.

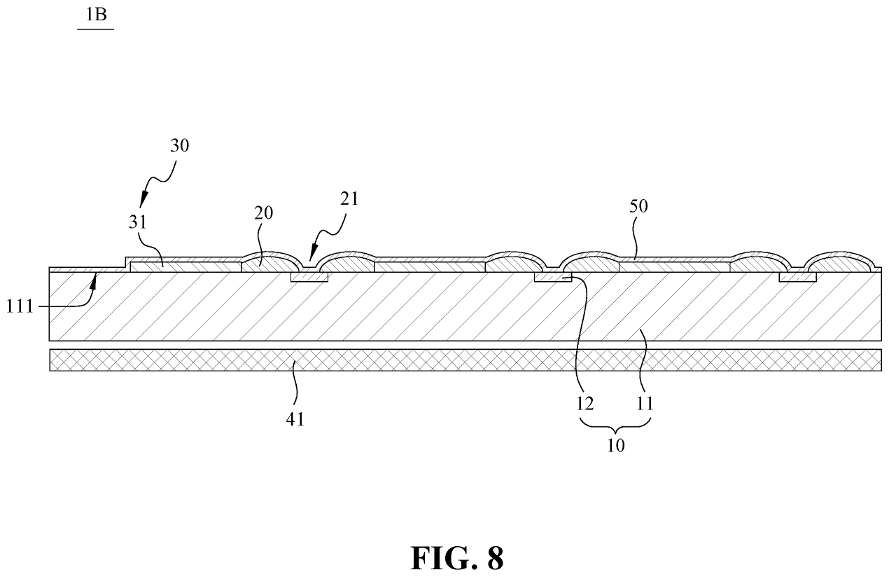

FIG. 8 is a schematic structural view of a third embodiment of the present invention. As shown in FIG. 8, the difference between the bioassay 1B of the third embodiment and the bioassay 1A of the second embodiment is that the bioassay 1B further includes a hydrophobic layer 50, and the hydrophobic layer 50 covers the outer surfaces of the microstructures 20, the top surfaces of the unit pixels 12, the outer surfaces of the first EWOD electrodes 31, and the exposed light-receiving surface 111. Thereby, the hydrophobicity of the hydrophobic layer 50 can increase the smoothness of the movement of the droplets 100 on the surface of the hydrophobic layer 50.

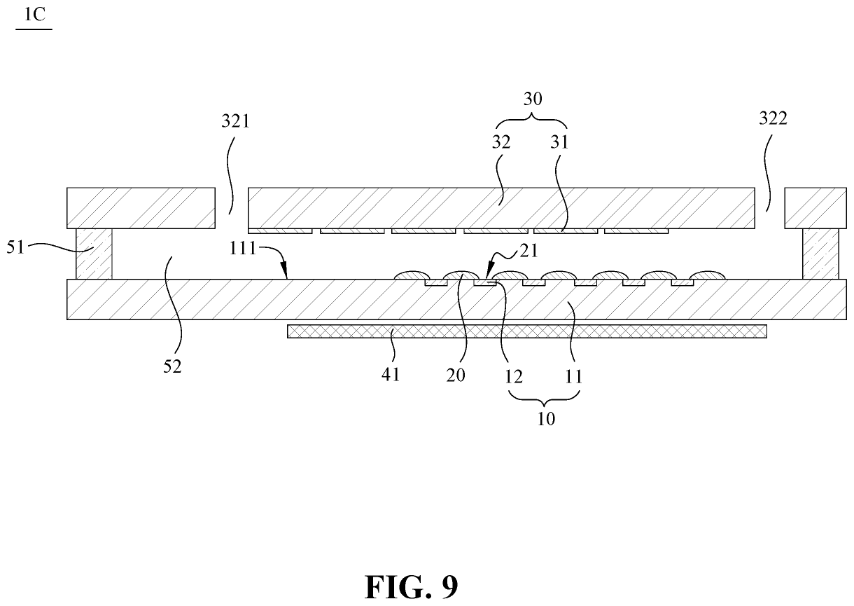

FIG. 9 is a schematic structural view of a fourth embodiment of the present invention. As shown in FIG. 9, the difference between the bioassay device 1C of the fourth embodiment and the bioassays 1, 1A, and 1B of the first to third embodiments is that: first, the first EWOD device 30 includes a first plate body 32, the first plate body 32 is disposed above the substrate 11 and defines an inlet 321 and an outlet 322, the first EWOD electrodes 31 are arranged on the bottom surface of the first plate body 32, and the outlet 322 is connected to a vacuum device (not shown); second, the bioassay 1C further includes a sealing layer 51, the sealing layer 51 is arranged between the first plate body 32 and the substrate 11, the sealing layer 51, the first plate body 32, and the substrate 11 jointly form a chamber 52, and the inlet 321 and the outlet 322 are respectively communicating with the chamber 52; third, the first EWOD electrodes 31, the unit pixels 12, and the microstructures 20 are all disposed in the chamber 52 and located between the inlet 321 and the outlet 322.

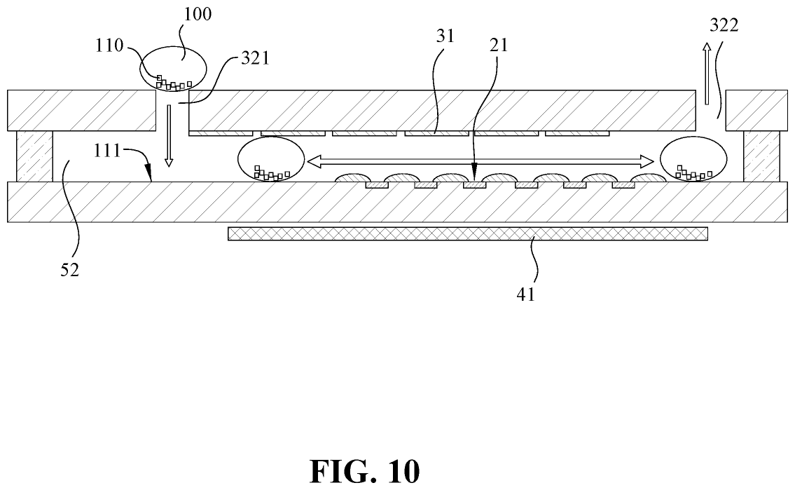

FIG. 10 is a schematic view of controlling the movement of the droplet 100 according to the fourth embodiment of the present invention. As shown in FIG. 10, the vacuum device evacuates the chamber 52 through the outlet 322 to generate a vacuum and provide a negative pressure. The droplets 100 containing a plurality of carriers 110 are attracted by the negative pressure and enter the chamber 52 from the inlet 321 and exit the chamber 52 from outlet 322. When the droplet 100 enters the chamber 52, the first EWOD electrodes 31 are energized, the first EWOD electrodes 31 generate an electrostatic force and control the droplets 100 to move back and forth on the light-receiving surface 111 by the electrostatic force.

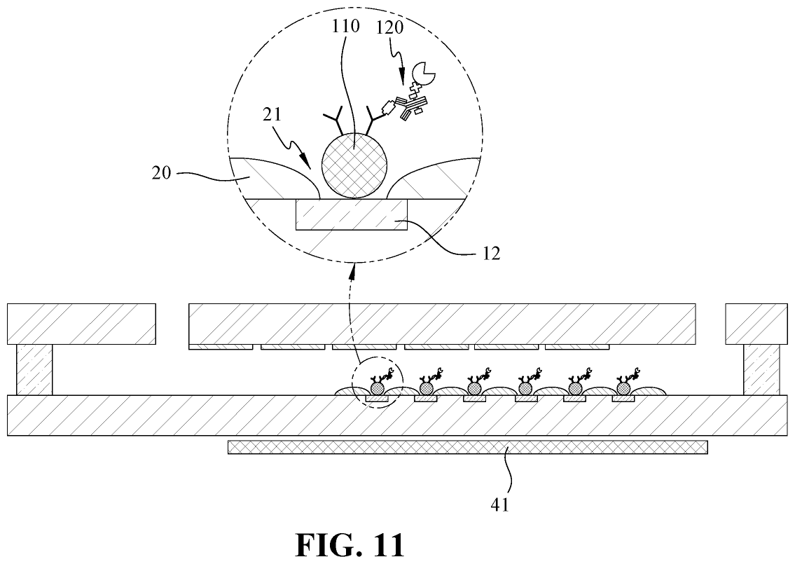

FIG. 11 is a schematic view of the carrier 110 evenly dispersed in the groove 21 of the fourth embodiment of the present invention. As shown in FIG. 10 and FIG. 11, during the process of the droplets 100 moving back and forth on the light-receiving surface 111, the droplets 100 will repeatedly pass through the grooves 21 for many times, so that the carriers 110 are evenly dispersed in the grooves 21, and each carrier 110 carries at least one biomolecule 120.

If the height of the chamber 52 is too high, the first EWOD electrodes 31 will be too far from the light-receiving surface 111, and the electrostatic force range of the first EWOD electrodes 31 will not be able to cover the light-receiving surface 111; thus, unable to control the droplets 100 to move. On the other hand, if the height of the chamber 52 is too low and the first EWOD electrodes 31 are too close to the light-receiving surface 111, it is difficult for the droplets 100 to move in the chamber 52.

Preferably, the height of the chamber 52 is controlled at 10-20 μm. Through experimental tests, this height enables the electrostatic force range of the first EWOD electrodes 31 to cover the light-receiving surface 111 and the droplets 100 can move smoothly in the chamber 52.

Preferably, the sealing layer 51 is cured by UV liquid glue irradiated with ultraviolet light. Therefore, the sealing layer 51 can combine and fix the first plate body 32 and the substrate 11, and provide a good sealing effect. More importantly, the bioassay 1C can easily achieve the effect of controlling the height of the chamber 52 by controlling the amount of UV liquid glue applied. However, the material of the sealing layer 51 is not limited thereto.

Preferably, the bioassay device 1C further includes a hydrophobic layer (not shown) covering the outer surfaces of the microstructures 20, the top surfaces of the unit pixels 12, and the exposed light-receiving surface 111. Thereby, the hydrophobicity of the hydrophobic layer can increase the smoothness of movement of the droplets 100 on the surface of the hydrophobic layer.

Compared with the bioassays 1, 1A, and 1B, because the bioassay 1C has a relatively closed structure, there are two disadvantages: First, the microstructures 20, the unit pixels 12, and the exposed light-receiving surface 111 are all hidden in the chamber 52, so that the attachments on the outer surfaces of the microstructures 20, the top surfaces of the unit pixels 12, and the exposed light-receiving surfaces 111 cannot be cleaned; therefore, the bioassay 1C cannot be cleaned. Second, the size of the droplet 100 is limited by the size of the inlet 321, which makes it difficult to control the size of the droplet 100. The size of the droplet 100 cannot be controlled to limit the number of carriers 110, and the number of the carriers 110 may exceed the number of the grooves 21. When the number exceeds the number of grooves 21, each groove 21 may have more than two carriers 110, that is, each unit pixel 12 will detect the incidence light of biomolecules 120 of more than two carriers 110, reducing the accuracy of determining the presence and/or concentration of biomolecules 120.

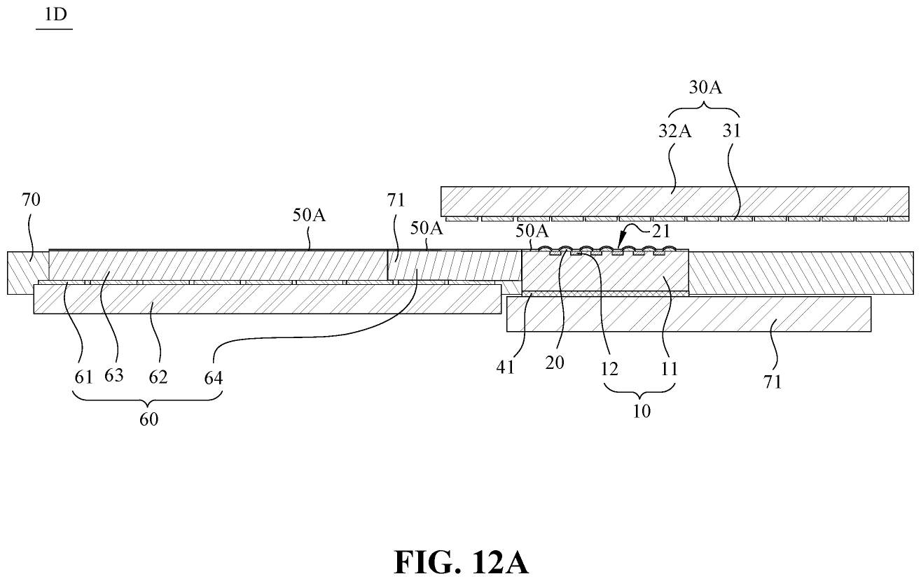

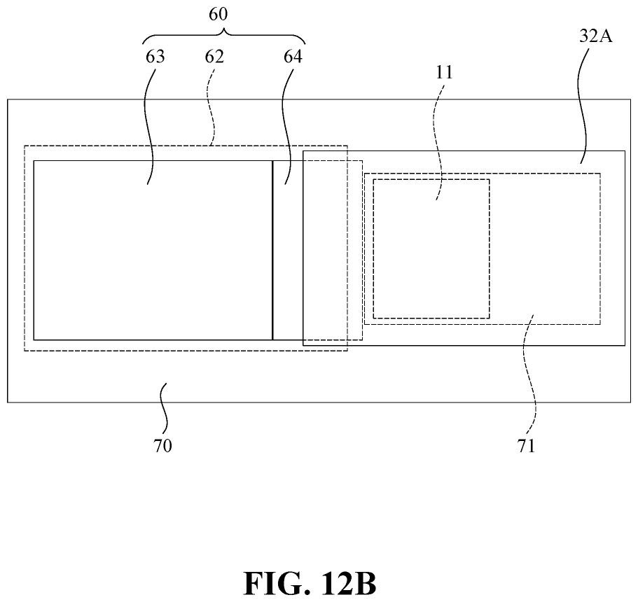

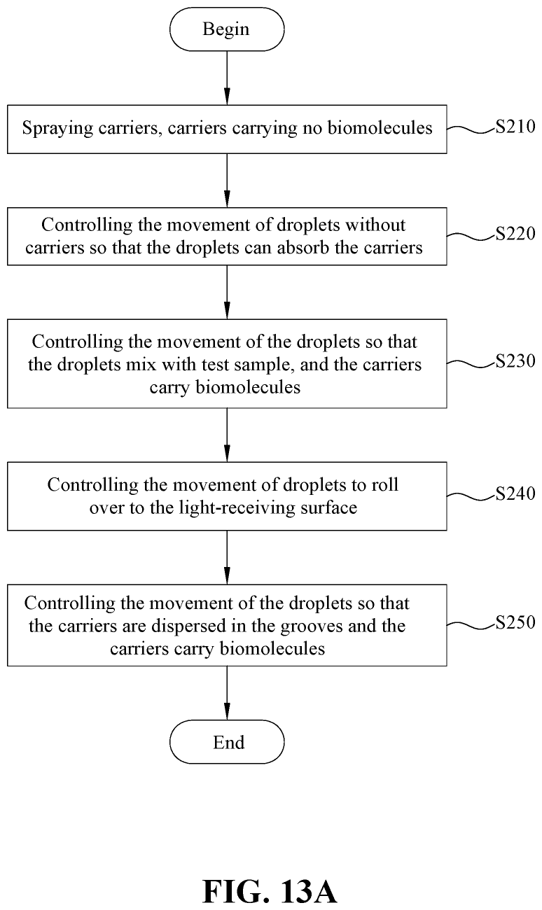

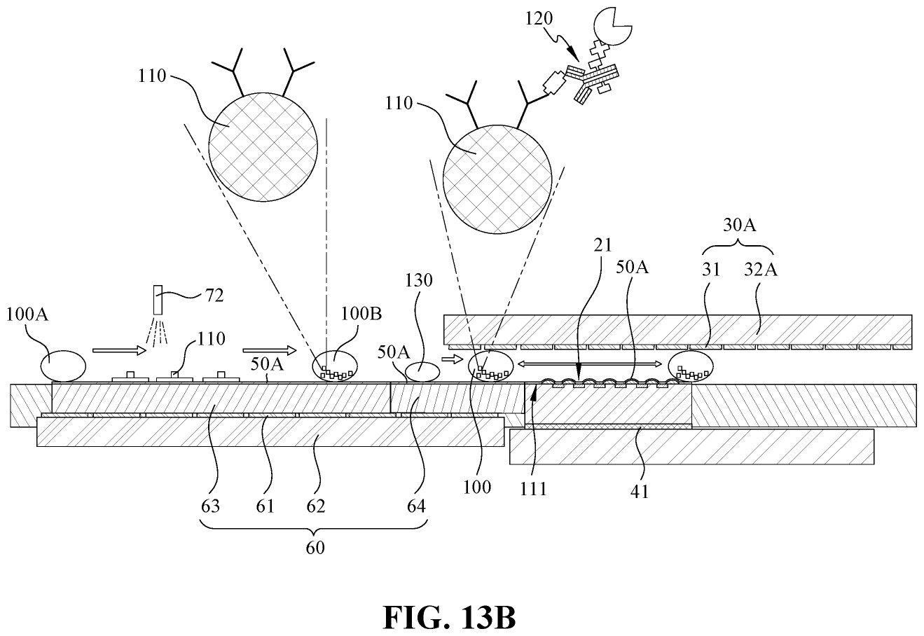

FIGS. 12A and 12B are a schematic structural view and a top view of the fifth embodiment of the present invention, respectively. As shown in FIGS. 12A and 12B, the difference between the bioassay 1D of the fifth embodiment and the bioassay 1C of the fourth embodiment is that: first, the bioassay 1D does not include a sealing layer, and the first plate body 32A has no inlet and outlet; second, the bioassay 1D further includes a second EWOD device 60. The second EWOD device 60 includes a plurality of second EWOD electrodes 61, a second plate body 62, a carrier tray 63, and a sample tray 64 for the sample to be tested. The second EWOD electrodes 61 are disposed on the top surface of the second plate body 62, the second plate body 62 is disposed on one side of the image sensing element 10; the carrier tray 63 and the sample tray 64 are both disposed above the second EWOD electrodes 61; the sample tray 64 is disposed between the carrier tray 63 and the image sensing element 10. The first EWOD device 30A extends above the sample tray 64. The carrier tray 63 is used to carry a plurality of carriers 110 and each carrier 110 carries no biomolecule 120 (see FIG. 13B), and the sample tray 64 is used for carrying a test sample 130 containing at least one biomolecule 120 (see FIG. 13B). Third, the bioassay 1D further includes a base 70 and a circuit board 71, and the image sensing element 10, the second EWOD device 60, and the magnet 41 are all arranged on the base 70. The circuit board 71 is arranged under the base 70 and is electrically connected to the unit pixels 12. The circuit board 71 has a microcontroller unit (MCU) and related circuits, and the circuit board 71 can also be used as a carrier for the substrate 11 of the image sensing device

FIGS. 13A and 13B are a flowchart and a schematic view of controlling the movement of droplets according to the fifth embodiment of the present invention, respectively. As shown in FIG. 13A and FIG. 13B, the process of controlling the movement of droplets in the fifth embodiment is as follows: in step S210, a nozzle 72 sprays a plurality of carriers 110 on the carrier tray 63 and each carrier 110 carries no biomolecule 120; in step S220, when the second EWOD electrodes 61 are energized, the second EWOD electrodes 61 generate an electrostatic force and control a droplet 100A containing no carrier 110 to move on the surface of the carrier tray 63, so that the droplet 100A adsorbs the carriers 110 to become a droplet 100B containing a plurality of carriers 110, each carrier 110 carries no biomolecule 120; in step S230, the second EWOD electrodes 61 further controls the droplet 100B containing the plurality of carriers 110 to move from the surface of the carrier tray 63 to the surface of the sample tray 64 by electrostatic force, so that the sample to be tested 130 is mixed with the droplet 100B containing the plurality of carriers 110, and each carrier 110 carries at least one biomolecule 120; in step S240, when the second EWOD electrodes 61 are powered off and the first EWOD electrodes 31 are powered on, the first EWOD electrodes 31 generate an electrostatic force, the first EWOD electrodes 31 further control the droplet 100 containing a plurality of carriers 110 to move from the surface of the sample tray 64 to the light-receiving surface 111 by electrostatic force; and in step S250, the first EWOD electrode 31 further controls the droplet 100 containing a plurality of carriers 110 to move back and forth on the light-receiving surface 111 by electrostatic force, so that the carriers 110 are evenly dispersed in the grooves 21, and each carrier 110 carries at least one biomolecule 120 .

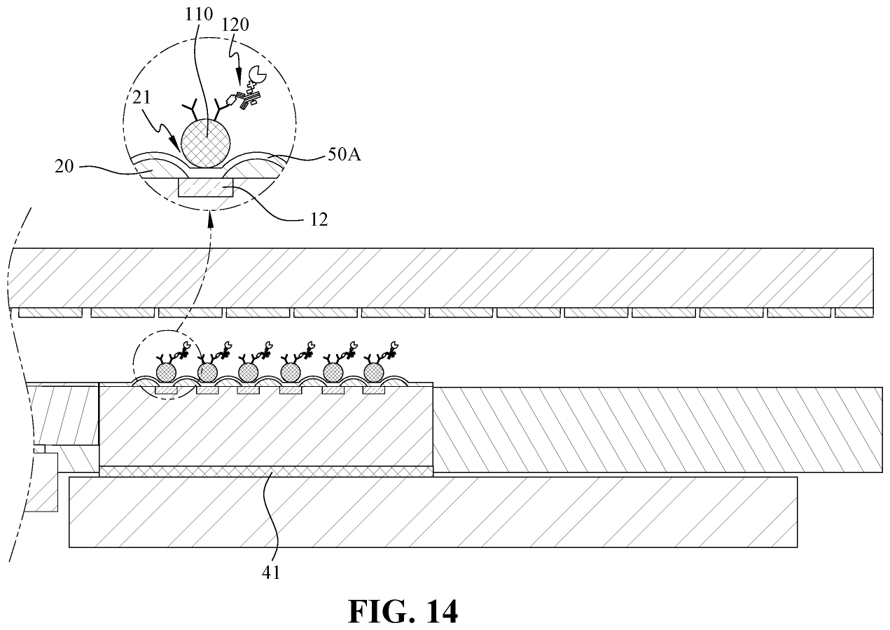

FIG. 14 is a schematic view of the carrier 110 evenly dispersed in the groove 21 of the fifth embodiment of the present invention. As shown in FIG. 13 and FIG. 14, during the process of the droplet 100 moving back and forth on the light-receiving surface 111, the droplet 100 will repeatedly pass through the grooves 21, so that the carriers 110 are evenly dispersed in the grooves in the grooves 21, and each carrier carries at least one biomolecule 120.

It should be noted that after the carrier 110 is combined with the biomolecule 120, the marker, fluorescent label, reporter molecule label or chemiluminescence label of the biomolecule 120 will start to emit light. Compared with the bioassays 1, 1A, 1B, and 1C, the bioassay 1D can perform the steps of spraying the carrier 110, the droplets 100 adsorbing the carrier 110, the carrier 110 binding the biomolecules 120, and dispersing the carrier 110 in the grooves 21 successively so that the biomolecules 120 enter the groove 21 along with the carrier 110 when the biomolecules 120 start to emit light, and the unit pixel 12 can immediately detect the incident light in the groove 21 and start to analyze the presence of the biomolecules 120 and/or concentration and high accuracy.

Furthermore, the structure of the bioassay 1D is relatively open, and has the following two advantages: first, the attachment on the top surface of the carrier tray 63, the top surface of the sample tray 64, the outer surfaces of the microstructures 20, the top surface of the unit pixels 12, and the exposed light-receiving surface 111 can be cleaned, so that the bioassay 1D can be reused; second, the number of carriers 110 contained is also easy to control and the size of the droplets 100 can be controlled so that the number of carriers 110 is lower than the number of grooves 21, and each groove 21 has at most one carrier 110, that is, each unit pixel 12 only detects the incident light of the biomolecules 120 of one carrier 110, which improves the accuracy of determining the presence and/or concentration of the biomolecules 120.

Preferably, the bioassay 1D further includes a hydrophobic layer 50A, and the hydrophobic layer 50A covers the top surface of the carrier tray 63, the top surface of the sample tray 64, the outer surface of the microstructures 20, the top surface of the unit pixel 12, and the exposed light-receiving surface 111. Thereby, the hydrophobicity of the hydrophobic layer 50A can increase the smoothness of movement of the droplets 100, 100A, 100B on the surface of the hydrophobic layer 50A.

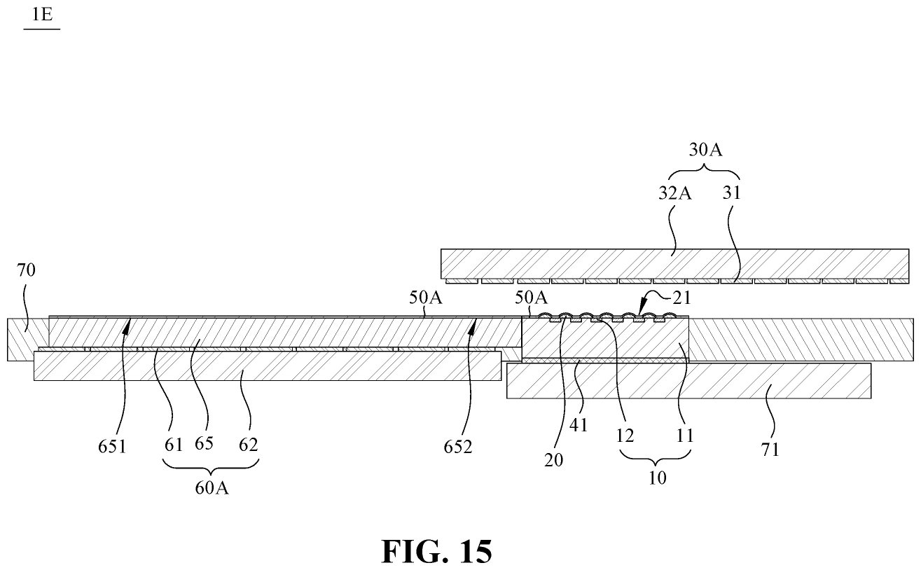

FIG. 15 is a schematic structural view of a sixth embodiment of the present invention. As shown in FIG. 15, the difference between the bioassay 1E of the sixth embodiment and the bioassay 1D of the fifth embodiment is that: first, the second EWOD device 60A includes the second EWOD electrodes 61, the second plate body 62 and a carrying tray 65, without the carrier tray and the sample tray; second, the carrying tray 65 is disposed above the second EWOD electrodes 61, and the carrying tray 65 has a carrier area 651 and a sample area 652, the sample area is disposed between the carrier area 651 and the image sensing element 10, the first EWOD device 30A extends above the sample area 652, and the carrier area 651 is used to carry a plurality of carriers 110 and each carrier 110 do not carry at least one biomolecule 120, the sample area 652 is used to carry a sample to be tested 130 containing at least one biomolecule 120, the hydrophobic layer 50A covers the top surface of the carrying tray 65, the outer surface of the structure 20, the top surface of the unit pixels 12, and the exposed light-receiving surface 111.

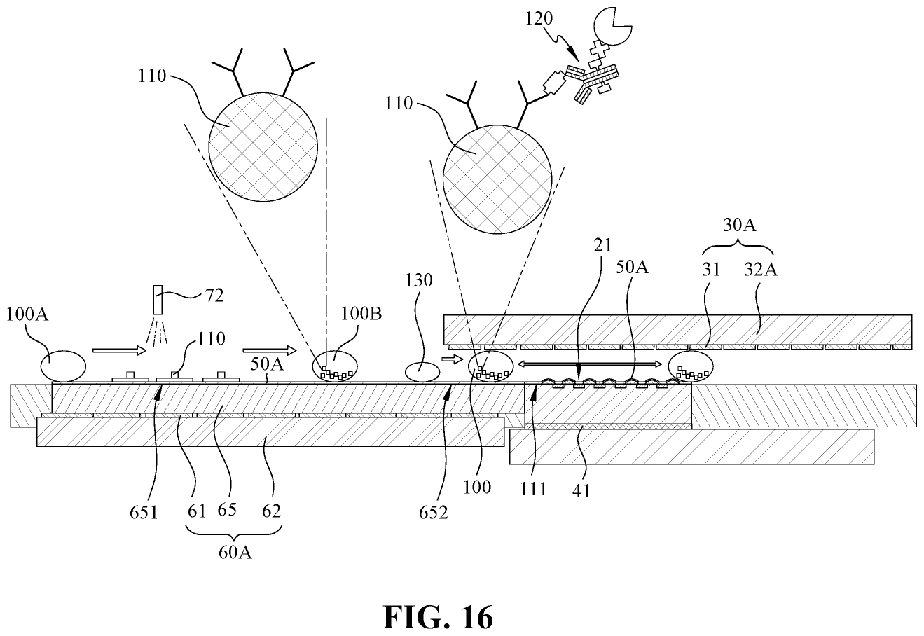

FIG. 16 is a schematic view of controlling the movement of droplets according to the sixth embodiment of the present invention. As shown in FIGS. 13A and 16, the process of controlling the movement of droplets in the sixth embodiment is as follows: in step S210, a nozzle 72 sprays a plurality of carriers 110 on the carrier area 651 and each carrier 110 carries no biomolecule 120; in step S220, when the second EWOD electrodes 61 are energized, the second EWOD electrodes 61 generate an electrostatic force and control a droplet 100A without the carrier 110 to move on the top surface of the carrier area 651 by the electrostatic force, so that the droplets 100A adsorb the carriers 110 to become a droplet 100B containing a plurality of carriers 110, each carrier 110 carries no biomolecule 120; in step S230, the second EWOD electrodes 61 further controls the droplet 100B containing the plurality of carriers 110 to move from the top surface of the carrier area 651 to the top surface of the sample area 652 by electrostatic force, so that the sample 130 is mixed with the droplet 100B containing the plurality of carriers 110, each carrier 110 carries at least one biomolecule 120; in step S240, when the second EWOD electrodes 61 are powered off and the first EWOD electrodes 31 are powered on, the first EWOD electrodes 31 generate an electrostatic force, the first EWOD electrodes 31 further control the droplet 100 containing a plurality of carriers 110 to move from the top surface of the sample area 652 to the light-receiving surface 111 by electrostatic force; and in step S250, the first EWOD electrode 31 further controls the droplet 100 containing a plurality of carriers 110 to move back and forth on the light-receiving surface 111 by electrostatic force, so that the carriers 110 are evenly dispersed in the grooves 21, and each carrier 110 carries at least one biomolecule 120.

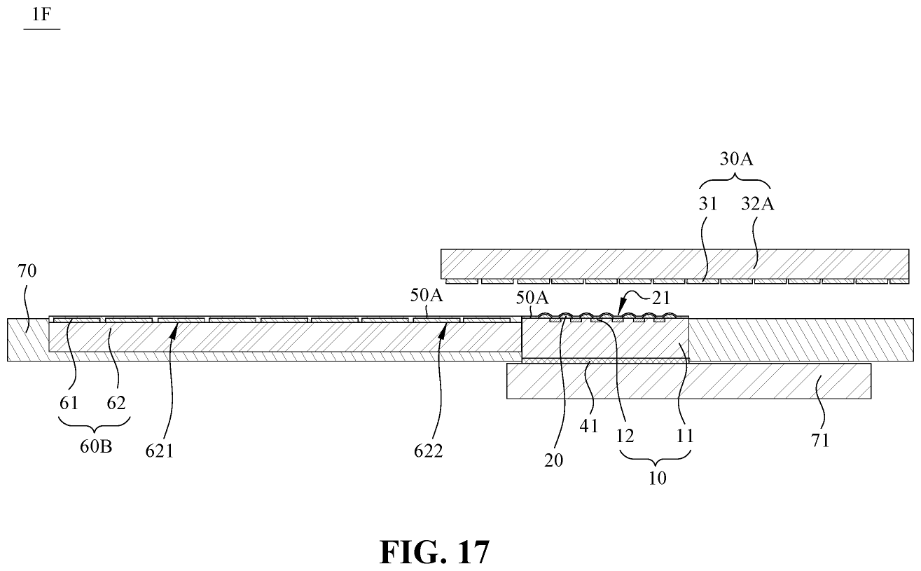

FIG. 17 is a schematic structural view of a seventh embodiment of the present invention. As shown in FIG. 17, the differences between the bioassay 1F of the seventh embodiment, the bioassay 1D of the fifth embodiment, and the bioassay 1E of the sixth embodiment are: first, the second EWOD device 60B has only the second EWOD electrodes 61 and the second plate body 62, without the carrier tray, the sample tray and the carrying tray; secondly, the second plate body 62 has a carrier area 621 and a sample area 622, the sample area 622 is disposed between the carrier area 621 and the image sensing element 10, the first EWOD device 30A extends above the sample area 622, and the carrier area 621 is used to carry a plurality of carriers 110 and each carrier 110 carries no biomolecule 120 , the sample area 622 is used to carry a sample to be tested 130 containing at least one biomolecule 120, the hydrophobic layer 50A covers the outer surfaces of the second EWOD electrodes 61, the top surface of the second plate body 62, the outer surface of the microstructures 20, the top surface of the unit pixels 12, and the exposed light-receiving surface 111.

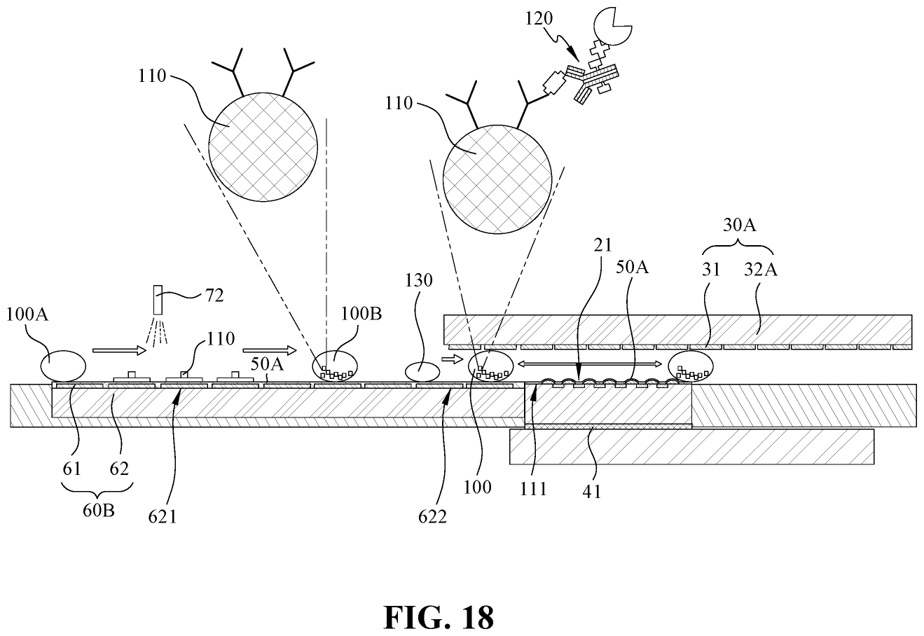

FIG. 18 is a schematic view of controlling the movement of droplets according to the sixth embodiment of the present invention. As shown in FIGS. 13A and 18, the process of controlling the movement of droplets in the seventh embodiment is as follows: in step S210, a nozzle 72 sprays a plurality of carriers 110 on the carrier area 621 and each carrier 110 carries no biomolecule 120; in step S220, when the second EWOD electrodes 61 are energized, the second EWOD electrodes 61 generate an electrostatic force and control a droplet 100A without the carrier 110 to move on the top surface of the carrier area 621 by the electrostatic force, so that the droplets 100A adsorb the carriers 110 to become a droplet 100B containing a plurality of carriers 110, each carrier 110 carries no biomolecule 120; in step S230, the second EWOD electrodes 61 further controls the droplet 100B containing the plurality of carriers 110 to move from the top surface of the carrier area 621 to the top surface of the sample area 622 by electrostatic force, so that the sample to be tested 130 is mixed with the droplet 100B containing the plurality of carriers 110, each carrier 110 carries at least one biomolecule 120; in step S240, when the second EWOD electrodes 61 are powered off and the first EWOD electrodes 31 are powered on, the first EWOD electrodes 31 generate an electrostatic force, the first EWOD electrodes 31 further control the droplet 100 containing a plurality of carriers 110 to move from the top surface of the sample area 622 to be tested to the light-receiving surface 111 by electrostatic force; and in step S250, the first The EWOD electrode 31 further controls the droplet 100 containing a plurality of carriers 110 to move back and forth on the light-receiving surface 111 by electrostatic force, so that the carriers 110 are evenly dispersed in the grooves 21, and each carrier 110 carries at least A biomolecule 120.

It is worth mentioning that, compared with the microfluidic chips, the structures of the bioassays 1-1F are easy to fabricate, and the cost is lower.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A bioassay with evenly dispersed carriers, comprising:
an image sensing element, further comprising: a substrate and a plurality of unit pixels, the substrate having a light-receiving surface, the unit pixels being disposed inside the substrate and close to the light-receiving surface, and each unit pixel having a photoelectric conversion element;
a plurality of microstructures, disposed on the light-receiving surface and forming a plurality of grooves, and the grooves being respectively located above the unit pixels; and
a first electrowetting-on-dielectric device, further comprising: a plurality of first electrowetting-on-dielectric (EWOD) electrodes, the first EWOD electrodes being disposed on the light-receiving surface and outside the grooves, or the first EWOD electrodes being disposed above the substrate.

2. The bioassay device according to claim 1, wherein the first EWOD device comprises a first plate body, the first plate body is disposed above the substrate, and the EWOD electrodes are arranged on a bottom surface of the first plate body.

3. The bioassay device according to claim 2, further comprising a second EWOD device, the second EWOD device comprising a plurality of second EWOD electrodes and a second plate body, the second EWOD electrodes being disposed on a top surface of the second plate body, the second plate body being disposed on one side of the image sensing element, and the first EWOD device extending to the above of the second EWOD device, the second EWOD device being used to carry a plurality of carriers and each of the carriers carrying no biomolecule, the second EWOD device being used to carry a sample to be tested containing at least one biomolecule.

4. The bioassay device according to claim 3, wherein the second EWOD device further comprises a carrier tray and a sample tray, and both the carrier tray and the sample tray are disposed on the above of the second EWOD electrodes, the sample tray is arranged between the carrier tray and the image sensing element, the first EWOD device extends to the above of the sample tray, and the carrier tray is used to carry a plurality of carriers and each of the carriers carries no biomolecule, the sample tray is used for carrying a test sample containing at least one biomolecule.

5. The bioassay device according to claim 3, wherein the second EWOD device further comprises a carrying tray, the carrying tray is disposed above the second EWOD electrodes, the carrying tray has a carrier area and a sample area, the sample area is arranged between the carrier area and the image sensing element, the first EWOD device extends above the sample area, the carrier area is for carrying a plurality of carriers, and the carriers carry no biomolecules, the sample area is used to carry a sample to be tested containing at least one biomolecule.

6. The bioassay device according to claim 3, wherein the second plate body has a carrying area and a sample area, and the sample area is arranged between the carrying area and the image sensing element, the first EWOD device extends above the sample area, the carrying area is used to carry a plurality of carriers and the carriers carry no biomolecule, the sample area is used to carry a test sample containing at least one biomolecule.

7. The bioassay device according to claim 3, further comprising a hydrophobic layer covering the top surface of the second EWOD device, the outer surfaces of the microstructures surface, the top surfaces of the unit pixels, and the exposed light-receiving surface.

8. The bioassay device according to claim 2, further comprising a sealing layer, the sealing layer being disposed between the first plate body and the substrate; the sealing layer, the first plate body, and the substrates jointly forming a chamber; wherein, the first plate body defining an inlet and an outlet, the inlet and the outlet being respectively communicating with the chamber, the first EWOD electrodes, the unit pixels, and the microstructures being all disposed in the chamber and located between the inlet and the outlet.

9. The bioassay device according to claim 1, wherein when the first EWOD electrodes are disposed on the light-receiving surface, the bioassay device further comprises a hydrophobic layer, and the hydrophobic layer covers the outer surfaces of the microstructures, the top surfaces of the unit pixels, the outer surfaces of the first EWOD electrodes, and the exposed light-receiving surface.

10. The bioassay device according to claim 1, further comprising at least one magnet, the at least one magnet is disposed inside or below the substrate, and has a magnetic force range covering the grooves.
